(19) United States
(12) Plant Patent Application Publication
(10) Pub. No.: US 2002/0059665 P1
(43) Pub. Date: May 16, 2002

(54) Strawberry plant named 'PLARIONFRE'

(76) Inventor: Jose Miguel Arias Lopez, Navarra (ES)

Correspondence Address: CHRISTIE, PARKER & HALE, LLP, 350 WEST COLORADO BOULEVARD, SUITE 500, PASADENA, CA, 91105, US

(21) Appl. No.: 09/796,024

(22) Filed: Feb. 28, 2001

(30) Foreign Application Priority Data
Jul. 28, 2000 (EP) 2000/1176

(51) Int. Cl. A01H 5/00

(52) U.S. Cl. PLT/208

(57) ABSTRACT

‘PLARIONFRE’ is a new variety of strawberry having inflorecence above the foliage and conical-shaped fruit.

### BACKGROUND OF THE INVENTION

The new variety of strawberry was created in a breeding program by crossing two parents; in particular, by crossing as seed parent an undistributed, unpatented variety internally designated 9244 and as pollen parent an undistributed and unpatented variety internally designated 86-032. Both parental varieties are proprietary and have not been commercialized or distributed.

The resulting seedling of the new variety was grown and asexually propagated by runners in Soria, Spain, 3° W., 41° N., 3,000 feet elevation. Clones of the new variety were further asexually propagated and extensively tested. This propagation and testing has demonstrated that the combination of traits disclosed herein which characterize the new variety are fixed and retained true to type through successive generations of asexual reproduction.

### SUMMARY OF THE INVENTION

The present invention relates to a new and distinct strawberry variety. Having the varietal denomination of the new variety ‘PLARIONFRE’. Among the characteristics which distinguish the new variety from other varieties of which I am aware are a combination of traits that include inflorescence which appears above the foliage and abundant production of red colored, conical shaped fruit.

### COMPARISON TO CLOSEST VARIETY

The new variety is closest to the variety Camarosa (U.S. Plant Pat. No. 8,708), but is distinguished therefrom by the following characteristics possessed by ‘PLARIONFRE’ which are different than, or not possessed by, ‘Camarosa.’

1. Camarosa exhibits more plant vigor than ‘PLARIONFRE.’

2. Camarosa exhibits more globose plant habit than ‘PLARIONFRE.’

3. Leaf surface undulation of Camarosa is weaker than ‘PLARIONFRE.’

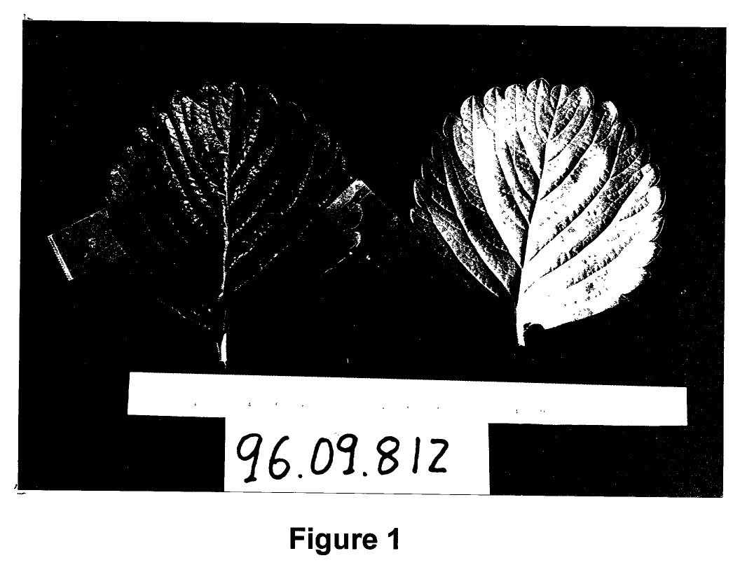

4. Camarosa has a darker green upperside leaf color (near 141 B to 141 A) than ‘PLARIONFRE’ (near 143 B to 143 A) . The differences in the leaves of Camarosa and the new variety are shown in FIGS. 1, 2, 3, and 4. These differences are maintained during the growing season.

5. In ‘PLARIONFRE’ the position of the inflorescence is above the foliage whereas in Camarosa, the inflorescence is level with the foliage.

6. Fruit of Camarosa are larger than ‘PLARIONFRE.’

7. Fruit of Camarosa is almost cylindrical in shape, whereas fruit of ‘PLARIONFRE’ is conical.

8. Fruit of Camarosa is dark red near 47B to 47A, whereas fruit of ‘PLARIONFRE’ is near 43A to 42A.

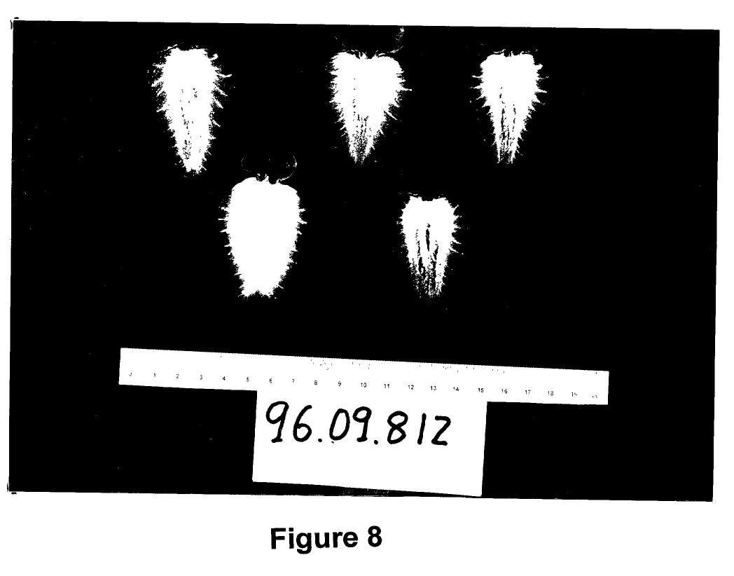

9. The differences in the fruit of Camarosa and of the new variety are shown in FIGS. 8, 9, 10, and 11. These differences are maintained during the harvest season.

10. Time of flowering for ‘PLARIONFRE’ is earlier than from Camarosa in the same location.

11. Time of fruit ripening for ‘PLARIONFRE’ is earlier than for Camarosa in the same location.

12. ‘PLARIONFRE’ fruit is less firm than Camarosa fruit.

### BRIEF DESCRIPTION OF ILLUSTRATIONS

The accompanying photographs show typical specimens of the new variety, designated 96.09.812 in the illustrations, including fruit, foliage and flower, in color as nearly true as it is reasonably possible to make in color illustrations of this character.

FIG. 1 shows the top and undersurface of a typical foliole of the new variety (designated 96.09.812) with the surface undulation of the upperside weaker and less dark green than the upperside of ‘Camarosa.’

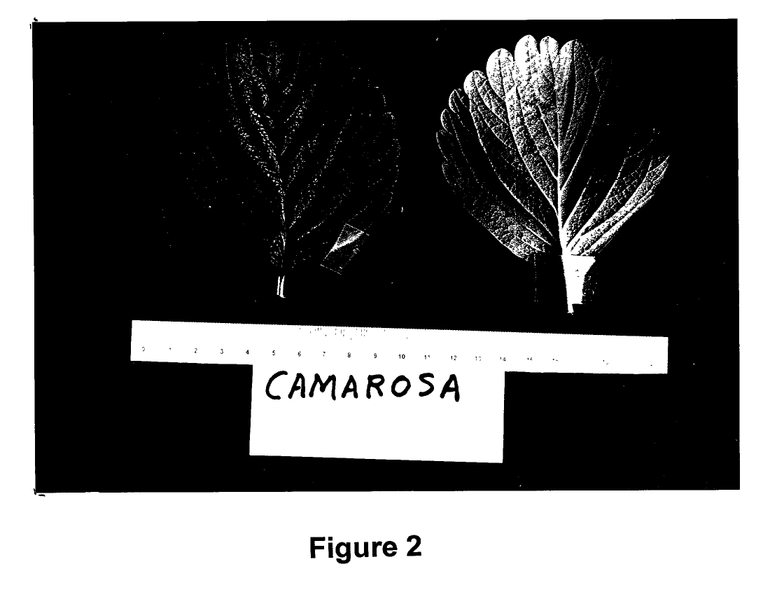

FIG. 2 shows the top and undersurface of a typical foliole of ‘Camarosa.’

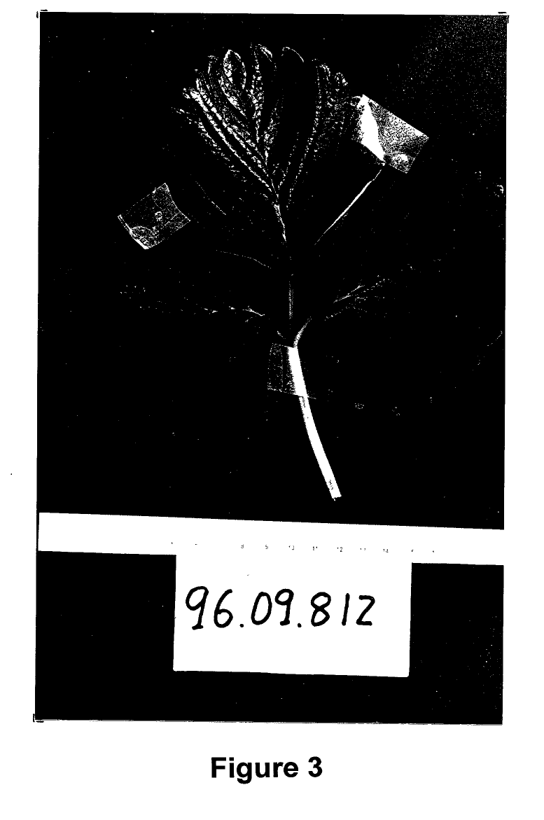

FIG. 3 shows a typical leaf of the new variety (designated 96.09.812) with more weak surface undulation of upperside and less dark green color of upperside than ‘Camarosa.’

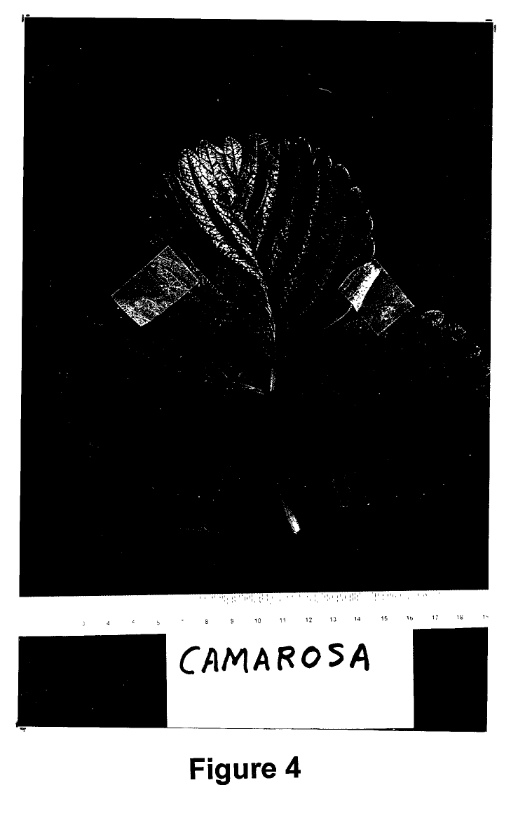

FIG. 4 shows a typical leaf of the ‘Camarosa.’

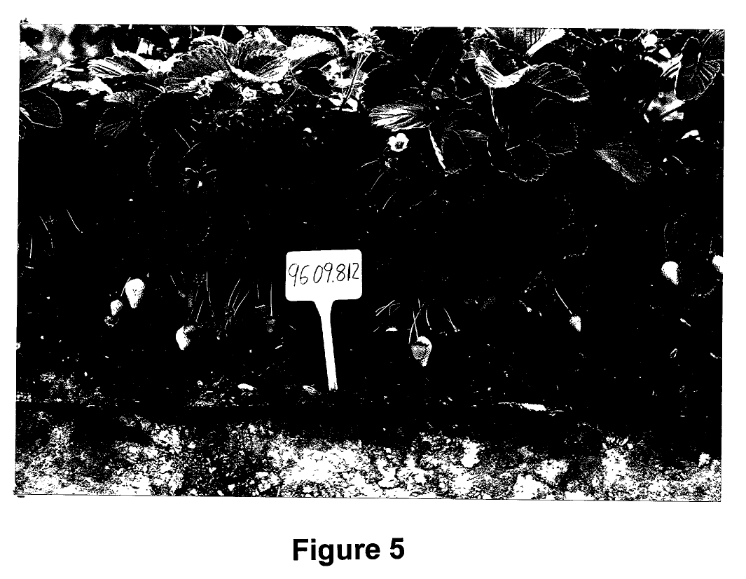

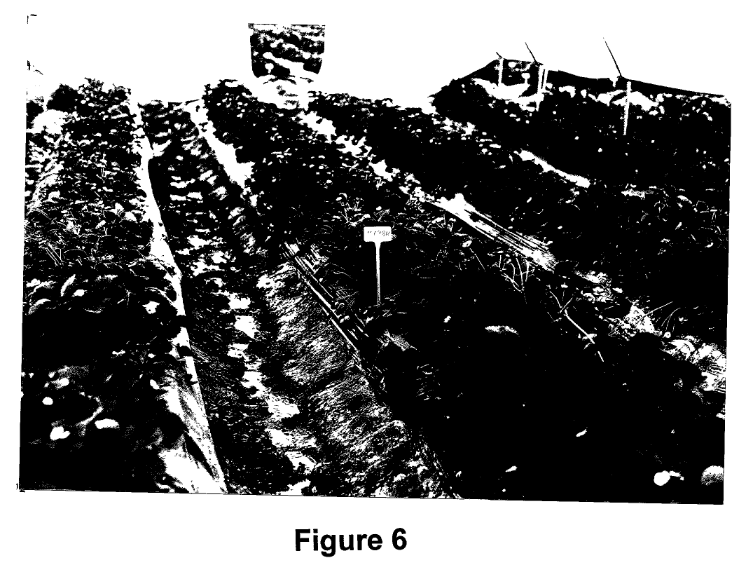

FIGS. 5 and 6 show the fruit against a background of the top surface of the foliage of the new variety (designated 96.09.812).

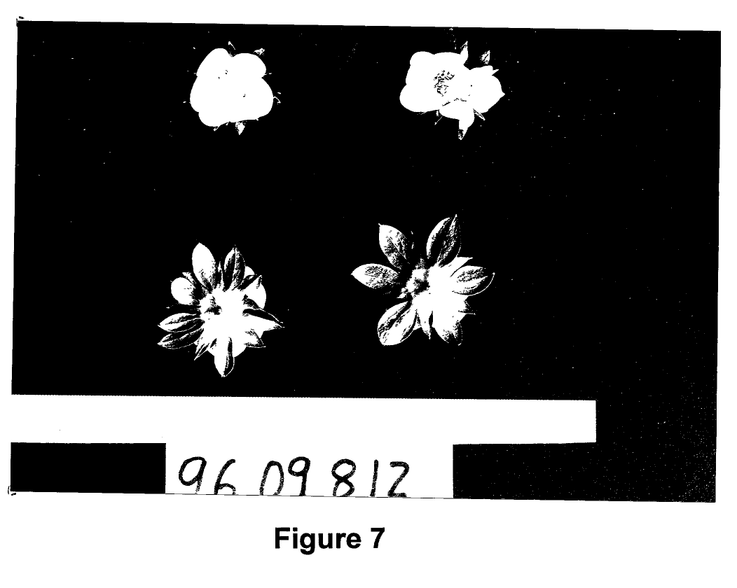

FIG. 7 shows the flower and reproductive organs of the new variety (designated 96.09.812).

FIG. 8 shows typical fruit of the new variety (designated 96.09.812) in cross section illustrating the typical flesh and flesh coloration, conspicuous core and core cavity and conical shape.

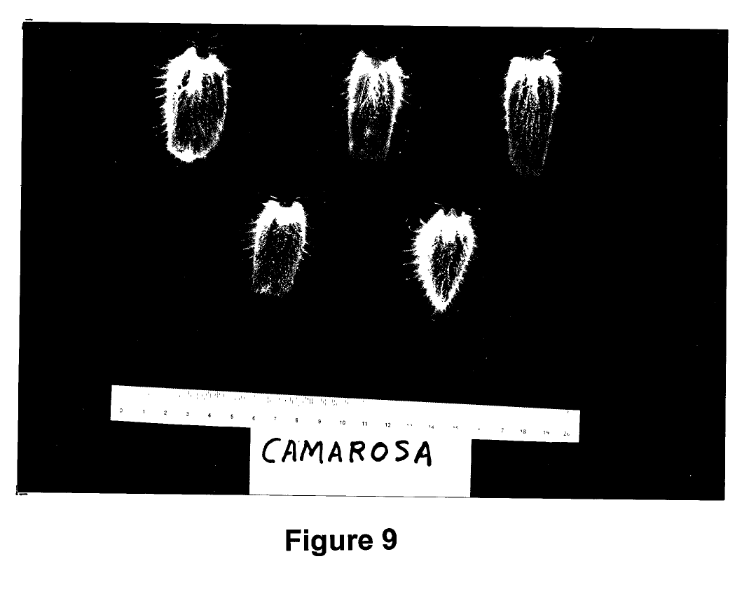

FIG. 9 shows typical fruit of the Camarosa in cross-section illustrating the typical flesh and flesh coloration, conspicuous core and core cavity and almost cylindrical shape.

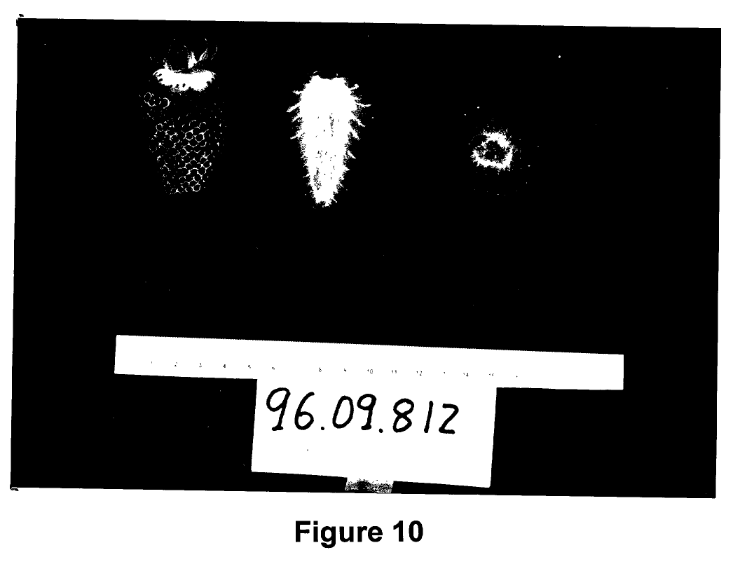

FIG. 10 shows whole and sliced, detached fruit of the new variety (designated 96.09.812) with the typical red color.

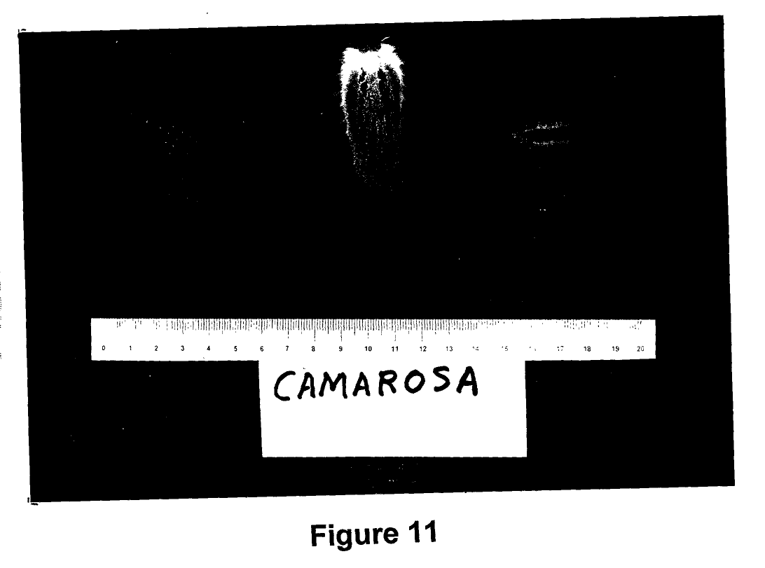

FIG. 11 shows whole and sliced, detached fruit of the Camarosa with the typical red dark color.

### DESCRIPTION OF THE NEW VARIETY

The following detailed description of the new variety is based upon observations taken of plants and fruits grown ‘underglass’, i.e. under tunnel, in the farm of La Mogalla in Cartaya (Huelva), Spain, 7° W., 37° N., 45 feet elevation.

The following description is in accordance with UPOV terminology and the color terminology herein is in accordance with The Royal Horticultural Society Colour Chart (RHS). The color descriptions and other phenotypical descriptions may deviate from the stated values and descriptions depending upon variation in environmental, seasonal, climatic and cultural conditions.

Propagation:

The new variety is principally propagated by way of runners. Although propagation by runners is presently preferred, other know methods of propagating strawberry plants may be used. Strawberries root well after transplanting.

The term ‘blistering’ used herein refers to the texture or rugosity or surface ondulation inherent to leaves and is generally a constant characteristic.

General:

‘PLARIONFRE’ is a short day variety that needs an induction to flowering by chilling, such as occurs at a high elevation nursery (fresh plant) or with cold storage (referred to as a frigo). Usually a short time is sufficient. ‘PLARIONFRE’ is self-fertile. It produces large quantity of pollen throughout the seasons and pollination is generally good as there are very few malformed fruit.

Production.—Plants described are from high elevation nursery in Soria, Spain (3° W., 41° N, 3,000 feet elevation) Fruit trials are from nursery in Cartaya (Huelva), Spain (7° W., 37° N, 45 feet elevation).

Date of planting.—Oct. 22, 1999.

Number of Repetitions.—2.

Plants per repetition.—375.

Comparison with Camarosa.—the new variety may be compared with Camarosa by reference to the Figures, e.g., FIG. 1 with FIG. 2; FIG. 3 with FIG. 4; FIG. 8 with FIG. 9; and FIG. 10 with FIG. 11.

Accumulated production of 1st quality fruit (g/plant)

| Variety | February 25^{th} | March 28^{th} | April 24^{th} | May 25^{th} |
|---|---|---|---|---|
| CAMAROSA | 49 | 266 | 521 | 706 |
| MILSEI | 52 | 269 | 446 | 558 |
| TUDNEW | 62 | 300 | 503 | 667 |
| PLARIONFRE | 130 | 399 | 583 | 729 |

| | 1^{ST} + 2^{ND} | | Weight |
|---|---|---|---|
| Variety | Quality Fruit | Total | (g/fruit) |
| CAMAROSA | 706 + 163 | 869 | 24-23 |
| MILSEI | 558 + 163 | 721 | 23-22 |
| TUDNEW | 667 + 131 | 798 | 24-23 |
| PLARIONFRE | 729 + 136 | 865 | 22 |

Production totals of
1st Quality Fruit (to May 25th) and
2nd Quality Fruit in g/plant

| | 1st | 2nd | TOTALS | % |
|---|---|---|---|---|
| Variety | quality | quality | (1st + 2nd) | quality |
| CAMAROSA | 706 | 163 | 869 | 19% |
| MILSEI | 558 | 163 | 721 | 23% |
| TUDNEW | 667 | 131 | 798 | 16% |
| PLARIONFRE | 729 | 136 | 865 | 16% |

$\% {2}^{nd} quality = \frac{ {2}^{nd} quality}{TOTAL} \times 100$

Weight (g/Fruit) at two dates
March 28^{th} and May 25^{th}

| Weight (g/fruit) | March 28^{th} | May 25^{th} |
|---|---|---|
| CAMAROSA | 24 | 23 |
| MILSEI | 23 | 22 |
| TUDNEW | 24 | 23 |
| PLARIONFRE | 22 | 22 |

Weight is shown as the average weight per fruit of First Quality Fruit.

FRUIT ANALYSIS

| | | PLARIONFRE | | |
|---|---|---|---|---|
| | CAMAROSA | (06.09.812) | CARTUNO | TUDNEW |
| Firmness | 0.46 | 0.19 | 0.36 | 0.28 |
| (KG) | | | | |
| Humidity & | 92.41 | 90.62 | 92.35 | 92.24 |
| Volatile | | | | |
| Matter (%) | | | | |
| Dry Matter | 7.59 | 9.38 | 7.65 | 7.76 |
| (%) | | | | |
| PH (to 20°) | 3.24 | 3.25 | 3.41 | 3.42 |
| Acidity as | 0.76 | 0.69 | 0.70 | 0.61 |
| Anhydride | | | | |
| Citric (%) | | | | |
| Soluble | 7.20 | 8.70 | 8.30 | 7.50 |
| Solids | | | | |
| (°Brix) | | | | |
| Maturity | 9.47 | 12.61 | 11.86 | 12.29 |
| Index | | | | |
| Content in | 610 | 770 | 570 | 610 |
| Ascorbic | | | | |
| Acid (ppm) | | | | |
| Dominant | 495 | 500 | 430 | 495 |
| Tonality | | | | |
| (nm) | | | | |
| Luminosity: | 15.30 | 17.20 | 26.00 | 31.80 |
| Transmit- | | | | |
| ance to | | | | |
| 460 nm | | | | |

The following definitions apply:

Firmness.—It is the fruit's resistance to penetration measured in Kilograms (Kg). The measure given has been obtained by the penetrometer ROZE Mod. Arbelette, with a 50 mm2 section head.

Dry Matter.—It is the weight of the residual left from the trituration of the fruit after the drying process at a temperature of 102° C.+2° C. until reaching constant weight.

(%) Dry Matter.—Weight Dry Matter/Weight Fresh Matter×100.

Humidity & Volatile Matter.—Represents the content in volatile matters and water of the fruits.

(%) Humidity & Volatile Matter.—100−% Dry Matter.

Maturity Index.—Relation between Soluble solids and Acidity as Anhydride Citric.

Maturity Index.—Soluble solids/Acidity as Anhydride Citric

Time of Ripening:

After planting as aforesaid, plants are grown in raised beds under-tunnel (small tunnel with small holes in plastic walls). Water and fertilizer were applied through drip irrigation. Time of ripening (50% of plants with ripe fruit) occurred about Jan. 12, 2000. First mature fruit was observed about Jan. 9, 2000, and maturity (15-20 gms/plant) is about January, 20.

General:

The growing period in Huelva, Spain, where the observations were made, is between about January 25^{th} and May 31^{st} of each year, with a maximum production at about mid-April. ‘PLARIONFRE’ is a variety that benefits from induction to flowering by chilling, usually a few hours are sufficient, preferably at temperatures of 7° C. or less. Normally, the minimum number of hours is accumulated in the field during several days.

Disease Resistance:

No particular sensitivity to any disease or parasite has been observed for ‘PLARIONFRE’.

The following additional information is provided to further describe the new variety:

| | |
|---|---|
| VARIETY | ‘PLARIONFRE,’ Breeder Ref. |
| | 96.09.812 |
| CLASSIFICATION | Fragaria, L. |
| PLANT | |
| Habit | Flat globose |
| Density | Open |
| Vigor | Medium |
| Height | About 17 cm. |
| Width | About 24 cm. |
| LEAF | |
| Upperside | Near 143B to 143A |
| Underside | Near 145D |
| Length | About 10 cm. |
| Width | About 11 cm. |
| Cross-section | Slightly concave |
| Leaf surface undulation | |
| or blistering | Weak |
| Number of leaflets | Three only |
| STEM CHARACTERISTICS | |
| Color | Near 145C |
| Position of hairs | upwards |
| Length | About 10 cm. |
| TERMINAL LEAFLET | |
| Length/width ratio | As long as broad |
| Length | About 6.5 cm. |
| Width | About 6.5 cm. |
| Shape of base | Obtuse |
| Shape of teeth | Obtuse |
| PETIOLE | |
| Position of hairs | Upwards |
| Length | About 10 cm. |
| STIPULE | |
| Anthocyanin coloration | Strong |
| STOLONS | |
| Number | Medium-about 7 |
| Anthocyanin coloration | Medium |
| Thickness | Medium, about 3 mm. |
| Pubescence | Medium |
| Color | Between near 145C to 144D |
| INFLORESCENCE | Position relative to foliage |
| | -Above |
| FLOWER | |
| Size | Medium |
| Size of calyx relative | Same size |
| to corolla | |
| Size of inner calyx | Larger |
| relative to outer | |
| Spacing of petals | Overlapping |
| FLOWER CHARACTERISTICS | |
| Diameter primary flowers | About 3.5 cm. |
| Diameter secondary | About 2.5 cm. |
| flowers | |
| Number of petals | Normally about 6-No |
| | significant fragrance |
| Time from bloom to | Between about 35 to 40 days |
| mature fruit | |
| (Huelva, Spain) | |
| PETAL | |
| Length/width ratio | Broader than long |
| FRUITING TRUSS | |
| Attitude | Semi-erect |
| FRUIT | |
| Ratio of length/maximum | Longer than broad |
| width | |
| Color | Between near 43A to 42A |
| Peduncle length of | Primary fruit between about 9 |
| inflorescence stem | to 11 cm., secondary fruit |
| | between about 6 to 7 cm., |
| | color near 145C |
| PRIMARY FRUIT | |
| Length | About 5.5 cm. |
| Width | About 4 cm. |
| SECONDARY FRUIT | |
| Length | About 4.5 cm. |
| Width | About 3 cm. |
| Size | Large |
| Predominant shape | Conical |
| Difference in shapes | Moderate |
| between primary and | |
| secondary fruits | |
| Band without achenes | Narrow |
| Unevenness of surface | Weak |
| Color | Between near 43A to 42A |
| Evenness of color | Slightly uneven |
| Glossiness | Strong |
| Insertion of achenes | Level with surface |
| Insertion of calyx | Level |
| Pose of the calyx | Reflexed |
| segments | |
| Size of the calyx in | Same size |
| relation to fruit | |
| diameter | |
| Adherence of calyx | Strong |
| Firmness | Firm |
| Color of flesh | Medium red-near 43A; |
| | lightening toward center |
| Evenness of color of | Even |
| flesh | |
| Sweetness | Medium |
| Acidity | Medium |
| Time of flowering | Early |
| Time of ripening | Very early |
| Time of bearing | Not remontant |
| Chilling | Weak |
| Planting date | October 22, 1999 |
| 10% flowering | November 29, 1999 |
| First mature fruits | January 9, 2000 |
| Maturity (15-20 | January 20, 2000 |
| gms/plant) | |

Time of Flowering Data:

Date of planting Oct. 22, 1999, in Cartaya (Huelva), Spain, about 7° W, 37° N, 45 feet elevation. 10% flowering occurred about Nov. 29, 1999, with first mature fruits about Jan. 9, 2000, and maturity (15-20 g/plant) about Jan. 20, 2000.

Time of flowers (50% of plants at first flower): observed about Dec. 3, 1999.

Storage Qualities:

‘PLARIONFRE’ fruit maintain their quality characteristics when keeping them in a frigo chamber at temperatures of about 2° C. during 48 hours. The fruit's color remains substantially the same.

I claim:

1. A new and distinct strawberry plant of the variety substantially as shown and described.
